(19) United States
(12) Patent Application Publication
(10) Pub. No.: US 2025/0084356 A1
(43) Pub. Date: Mar. 13, 2025

(54) PHYSIOLOGICALLY-RELEVANT, SERUM-FREE IN VITRO ANGIOGENESIS PLATFORM

(71) Applicant: South Dakota Board of Regents, Pierre, SD (US)

(72) Inventors: Laura A.E. Brunmaier, Rapid City, SD (US); Travis W. Walker, Rapid City, SD (US); Cyle Miller, Saint Onge, SD (US); Timothy M. Brenza, Rapid City, SD (US)

(73) Assignee: South Dakota Board of Regents, Pierre, SD (US)

(21) Appl. No.: 18/725,006

(22) PCT Filed: Jan. 6, 2023
(86) PCT No.: PCT/US23/60256

§ 371 Date: Jun. 27, 2024

Related U.S. Application Data
(60) Provisional application No. 63/297,609, filed on Jan. 7, 2022.

(51) Int. Cl. C12M 3/00; C12M 1/00

(52) U.S. Cl. CPC C12M 21/08; C12M 29/00; C12M 29/10; C12M 41/14

(57) ABSTRACT

A custom platform is developed that is capable of culturing endothelial cells three-dimensionally while inducing physiological stimuli on the cells. A model includes serum-free conditions, allowing more quantifiable angiogenesis studies that investigate the dose dependent effects of various growth factors. Angiogenesis plays a critical role during development, wound healing, and disease; therefore, further investigation of the pathway has broad implications. Also, the development of in vitro vascularized tissue models via angiogenesis provides the ability to investigate endothelial responses after exposure to nanoparticles and mechanisms of drug delivery.

### CROSS-REFERENCE TO RELATED APPLICATIONS

This application claims priority under 35 U.S.C. § 119 to provisional application Ser. No. 63/297,609 filed Jan. 7, 2022, which is incorporated by reference in its entirety.

### FIELD OF THE INVENTION

The present invention relates to a physiologically-relevant, serum-free in vitro angiogenesis model with quantifiable exposure of growth factors. More particularly, but not exclusively, the present invention relates to a physiologically-relevant, serum-free in vitro angiogenesis platform.

### BACKGROUND

The current standard for investigating fundamental biological mechanisms and performing biomedical testing is cell culture and animal models. Two-dimensional cell culture has been pivotal in advancing our understanding of biological processes; however, cellular response in a 2D environment often differs from an in vivo response. The purpose of the animal model is to serve as a medium for investigating the interactions between a complex system with a device, drug, or biological modification, which may overcome the challenges that are discussed in 2D cell culture. However, evidence indicates that the results from animal models cannot confidently predict human outcomes either. The challenge behind this extrapolation is attributed to genetic, immunologic, and cellular differences between animals and humans. Therefore, there is a need for the development of standardized, physiologically-relevant, in vitro models using human cells that may provide a reservoir for data collection surrounding responses in human cells.

### SUMMARY

At least one object, feature or advantage of the present disclosure is to provide a device with a platform for setting a material into the cylindrical compartment, having one or more perfusable channels, installing into a flow loop if necessary, seal the compartment housing the material, and allow for microscopic imaging. The device is machined out of aluminum. It includes a low durometer material (o rings or silicone) to seal the inner housing compartment and either bolts to apply compression or having the cylindrical compartment come together around the soft durometer material or o rings and fastened by threading. Tracks are in place to guide syringes into and out of the material compartment that both set channels in the material and act as part of the flow loop to pass fluid or other materials into and out of the cylindrical housing area. These tracks can be locked into place at a desired position to inhibit them from moving into or out of the cylindrical compartment when it is not intended.

Another object, feature or advantage of the present disclosure is to provide glass coverslips used on both the top and the bottom of the device to allow light to pass through when on a microscope making the sealed device microscope compatible while maintain flow conditions.

A further object, feature or advantage of the present disclosure is to provide a platform wherein the distance between the syringes that pass through the device are at strictly 1 mm apart. Depending on the desired application, one channel can be removed by removing two of the syringes resulting in one channel that passes through the material as opposed to two parallel.

A still further object, feature or advantage of the present disclosure is to provide a platform where material compatible with device is only limited to material that can be cast into the cylindrical channel.

One further object, feature or advantage of the present disclosure is to provide a custom designed stage was 3D printed to secure the device on the microscope stage which not interrupting flow in the device.

Another object, feature or advantage of the present disclosure is to provide a device modeled around biological systems for modeling in vivo systems more accurately in vitro. However, use can be expanded to any system where flow occurs through some type of medium.

Other objects, features or advantages of the present disclosure include, for example, setting biomimetic hydrogels such as collagen and fibrin, seeding cells into the channel and allowing them to attach and proliferate. It also includes by way of example, introducing other materials like nanoparticles or drugs into the system and investigating the biological effects and delivery mechanisms.

Still other objects, features or advantages of the present disclosure include, for example, removing one channel for investigating the embedding of organoids or spheroids into the hydrogel, effects of diffusion or interstitial flow of chemotactic agents through the hydrogel, and effects of these agents on cells seeded in the channel perfusing the hydrogel.

In at least one exemplary aspect, a physiologically-relevant, serum-free in vitro angiogenesis platform is disclosed. The platform includes, for example, a cell chamber, one or more perfusable channels disposed within the cell chamber, one or more ports in a sidewall of the cell chamber, a track operably disposed on at least one side of the cell chamber, and at least one connector disposed in the track. The at least one connector may be configured for introducing one or more concentrations of nanoparticles into the cell chamber.

In at least one other exemplary aspect, a system for a physiologically-relevant, serum-free in vitro angiogenesis platform is disclosed. The system includes, for example, an incubator operably configured for housing a cell media, a peristaltic pump for moving the cell media, a microscope for conducting microscopy of the cell media, a cell chamber having one or more perfusable channels, a viewing window for observation with the microscope, and one or more ports in a sidewall of the cell chamber for introducing one or more concentrations of nanoparticles. Endothelial cells may line the one or more perfusable channels.

In still another exemplary aspect, a method for physiologically-relevant, serum-free in vitro angiogenesis is disclosed. The method includes by way of example, such steps as providing a cell chamber having one or more perfusable channels, a viewing window for microscopy observation, and one or more ports in a sidewall of the cell chamber, introducing cell media into the cell chamber, introducing one or more concentrations of nanoparticles into the cell chamber via the one or more ports in the sidewall of the cell chamber, and circulating cell media through the cell chamber.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

### BRIEF DESCRIPTION OF THE DRAWINGS

Illustrated embodiments of the disclosure are described in detail below with reference to the attached drawing figures, which are incorporated by reference herein.

FIG. 1 is a pictorial representation of a flow loop with capabilities for microscopy in accordance with an exemplary aspect of the present disclosure.

FIG. 1A is pictorial representation of a development of an in vitro vascularized tissue model, with or without physiological flow where cells are cultured into one of the collagen channels in accordance with an exemplary aspect of the present disclosure.

FIG. 1B is pictorial representation of a development of an in vitro vascularized tissue model, with or without physiological flow where once confluent, VEGF is introduced into the parallel channel in accordance with an exemplary aspect of the present disclosure.

FIG. 1C is pictorial representation of a development of an in vitro vascularized tissue model, with or without physiological flow where VEGF diffuses through the gel, interacting with the ECs in the parallel channel and ECs begin sprouting into and navigating the hydrogel medium in accordance with an exemplary aspect of the present disclosure.

FIG. 1D is pictorial representation of a development of an in vitro vascularized tissue model, with or without physiological flow where chemotaxis guides the ECs through the hydrogel, eventually reaching the VEGF channel and successful anastomosis leads to an in vitro vascularized tissue model in accordance with an exemplary aspect of the present disclosure.

FIG. 2A is a pictorial representation depicting cells that have been flowed into the channels before the flow was stopped to allow the cells to attach to the hydrogel walls in accordance with an exemplary aspect of the present disclosure.

FIG. 2B is a pictorial representation depicting cells that are adherent and confluent in accordance with an exemplary aspect of the present disclosure.

FIG. 2C is a pictorial representation depicting commencement of flow, where chemicals or particles of interest can be introduced to the cells from the flow stream in accordance with an exemplary aspect of the present disclosure.

FIG. 3 is CAD exploded view of an exemplary model in accordance with at least one aspect of the present disclosure.

FIG. 4 is CAD collapsed view of an exemplary model in accordance with at least one aspect of the present disclosure.

FIG. 5A is a pictorial representation of a hydrogel compartment or cell chamber in accordance with an exemplary aspect of the present disclosure.

FIG. 5B is a pictorial representation of syringe paths passing through the sides of the device and the slip in accordance with an exemplary aspect of the present disclosure.

FIG. 5C is a pictorial representation of a machined channel showing on the top of the aluminum slip housing one of the O-rings for compressing and sealing the assembled device.

FIG. 6 is a pictorial illustration of mold in accordance with an exemplary aspect of the present disclosure.

FIG. 7 is a pictorial illustration showing an exploded view of the mold in accordance with an exemplary aspect of the present disclosure.

FIG. 8 is a pictorial illustration of the exemplary model in accordance with at least one aspect of the present disclosure.

FIG. 9 is a pictorial illustration of the flow tubing of the chamber in accordance with an exemplary aspect of the present disclosure.

FIG. 10 is a pictorial illustration of the chamber in accordance with an exemplary aspect of the present disclosure.

FIG. 11 is another pictorial illustration of the chamber and flow tubing in accordance with an exemplary aspect of the present disclosure.

FIG. 12 is another pictorial illustration of the chamber in accordance with an exemplary aspect of the present disclosure.

FIG. 13 is another pictorial illustration of the chamber and flow tubing in accordance with an exemplary aspect of the present disclosure.

FIG. 14 is a flowchart illustrating a method of for physiologically-relevant, serum-free in vitro angiogenesis in accordance with an exemplary aspect of the present disclosure.

### DETAILED DESCRIPTION

The present disclosure is directed to a physiologically-relevant, serum-free in vitro angiogenesis model with quantifiable exposure of growth factors. The disclosure includes, for example, a model 30 to measure an inflammatory response of endothelial cells that are exposed to nanoparticles, as well as efficacy and mechanisms of nanoparticle drug delivery.

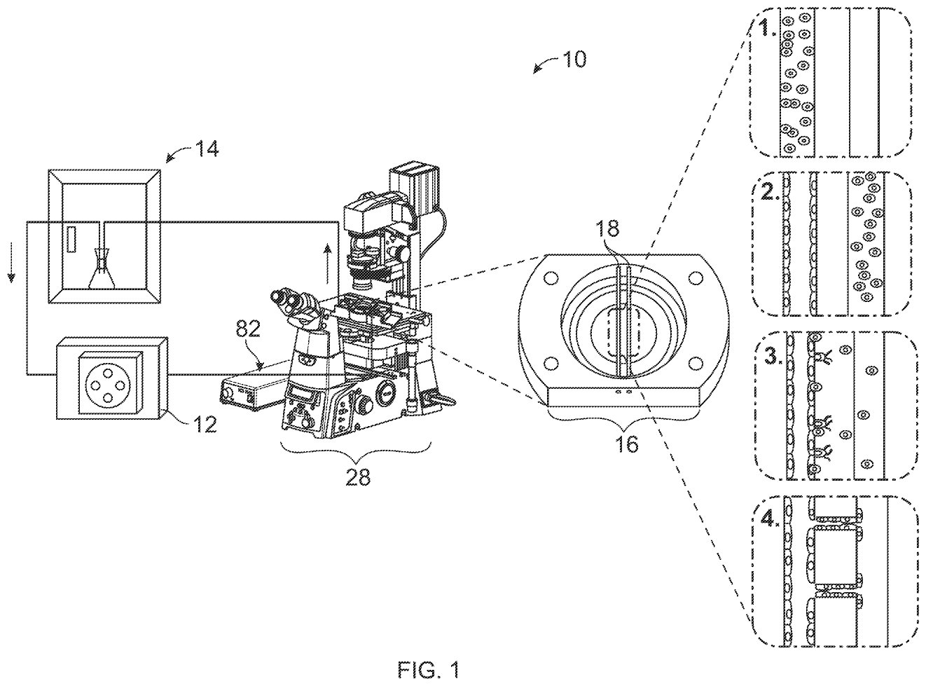

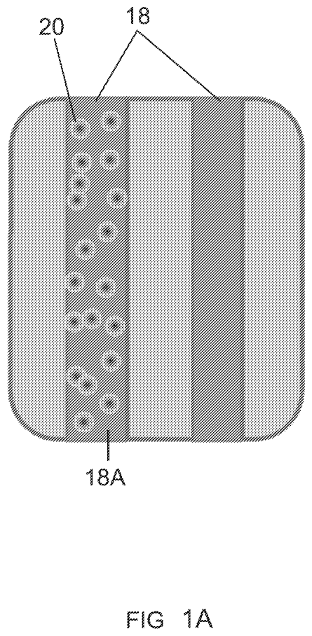

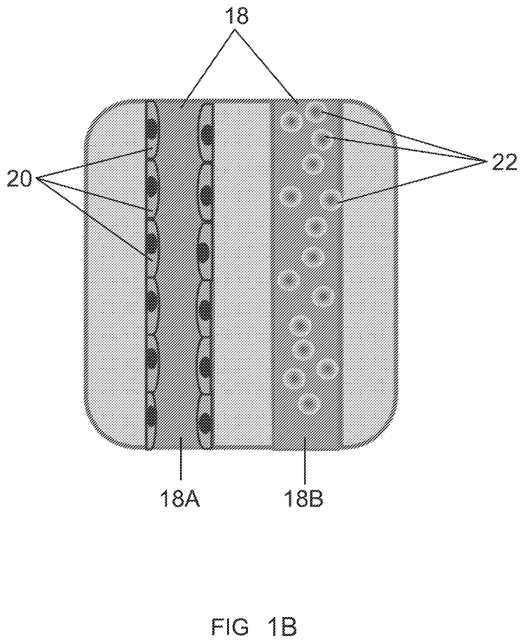

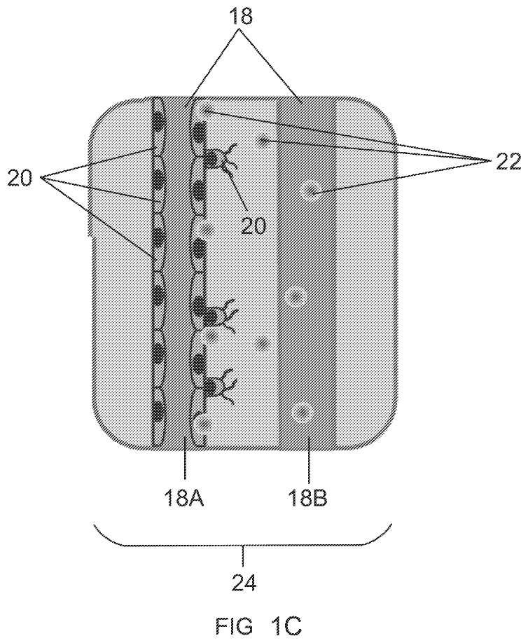

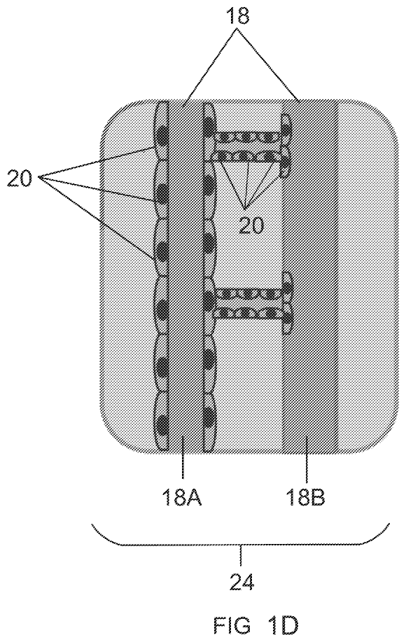

In at least one example of the system 10, a peristaltic pump 12 drives media that is housed in an incubator 14 at 5% CO₂ and 37° C. The temperature and percent CO₂ may be higher or lower in other aspects of the present disclosure. The media travels through the cell chamber 16, where endothelial cells 20 (ECs) line the hydrogel channels 18 and returns to the media reservoir completing the loop (FIG. 1), a microscope 28 may be used to view the cell chamber 16. A controller 82 may be operatively connected to the microscope, peristaltic pump 12, incubator 14, and the device 30 to control one or more aspects of the system such as movement of materials, cell media, or EC's 20 through the system. The exploded views shown in FIGS. 1A-1D detail the cell chamber 16 and the hydrogel channels 18. For example, FIG. 1 is a pictorial representation of a flow loop with capabilities for microscopy in accordance with an exemplary aspect of the present disclosure. FIG. 1A is pictorial representation of a development of an in vitro vascularized tissue model, with or without physiological flow where cells 20, such as ECs, are cultured into one of the collagen channels 18A of a plurality of channels 18 in accordance with an exemplary aspect of the present disclosure. FIG. 1B is pictorial representation of a development of an in vitro vascularized tissue model, with or without physiological flow where once confluent, vascular endothelial growth factor (VEGF) 22 is introduced into the parallel channel 18B in accordance with an exemplary aspect of the present disclosure. FIG. 1C is pictorial representation of a development of an in vitro vascularized tissue model, with or without physiological flow where VEGF 22 diffuses through the gel 24, interacting with the ECs 20 in the parallel channel and ECs 20 begin sprouting into and navigating the hydrogel medium 24 in accordance with an exemplary aspect of the present disclosure. FIG. 1D is pictorial representation of a development of an in vitro vascularized tissue model, with or without physiological flow where chemotaxis guides the ECs 20 through the hydrogel, eventually reaching the VEGF channel 18B and successful anastomosis leads to an in vitro vascularized tissue model in accordance with an exemplary aspect of the present disclosure.

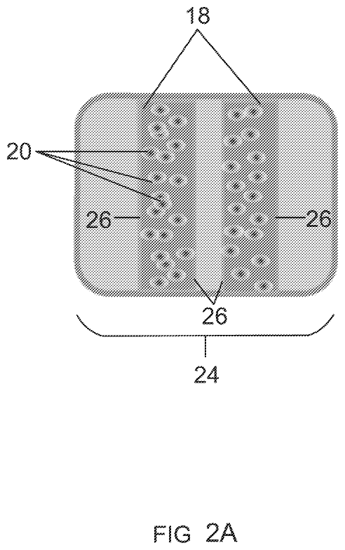

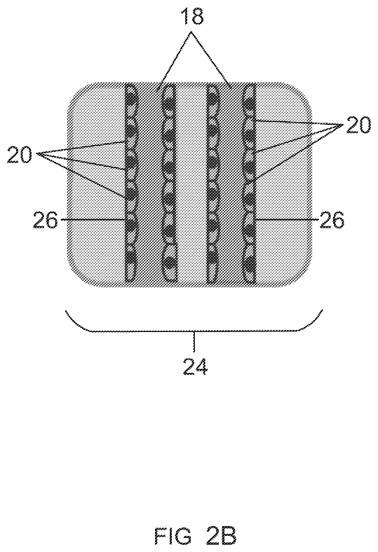

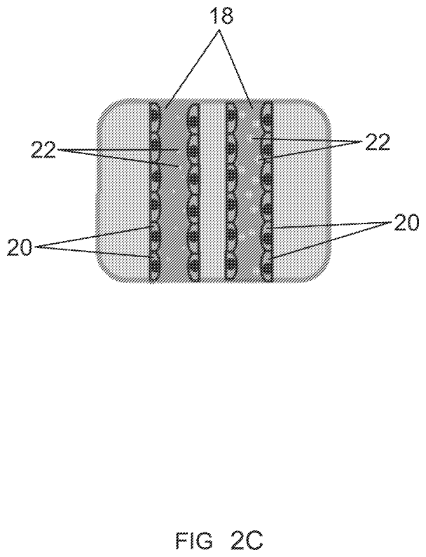

FIG. 2A is a pictorial representation depicting cells 20 that have been flowed into the channels 18 before the flow was stopped to allow the cells to attach to the hydrogel walls 26 of the channels 18 in accordance with an exemplary aspect of the present disclosure. FIG. 2B is a pictorial representation depicting cells 20 that are adherent and confluent in accordance with an exemplary aspect of the present disclosure. FIG. 2C is a pictorial representation depicting commencement of flow, where chemicals or particles of interest can be introduced to the cells from the flow stream in accordance with an exemplary aspect of the present disclosure.

### Exemplary Design Criteria for the In Vitro System

To design and engineer the in vitro system, examples of critical design criteria that can be required included that the device 30 needed to house a 3D hydrogel 24, to be fully enclosed, to allow incubation, to be microscope 28 compatible, to contain cylindrical channels 18, to control their separation distance, to control both flow rates, to control acute exposure, to control environmental conditions, to be reusable and autoclavable, and to allow local sampling, while being customizable.

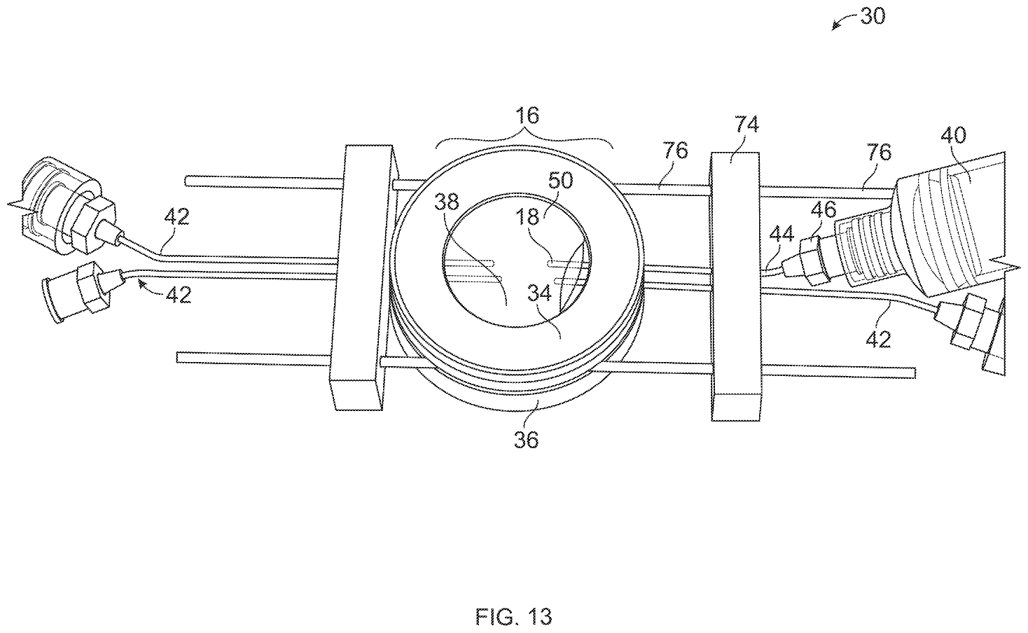

The device may provide a sealable chamber 16 or cast with a sealed topside 34 and a sealed bottom side 36, as shown in FIG. 13. The sealable chamber 16, the sealed top side 34, or the sealed bottom side 36 may incorporate a transparent material 38 to allow light to pass through for microscopy or imaging during static or microfluidic-flow conditions. Hollow channels 18 may pass through the body of the chamber 16 to provide internal access for applications, such as allowing a path for channels of a cast material. The channels 18 may be accessible via a smaller long probe 40 or syringe 40 to sample the space within the cast material or to provide a local stimulation within the channel 18. The branching point 42 of the syringe or channel probing device 40 may provide access to the channels within the chamber that branches from the flow stream in one or more points. This device 30 is useful in 3D cell-culture applications. Such models include angiogenesis, drug delivery, drug development, tissue engineering, materials characterization, organoid/spheroid development, ex vivo tissue experiments, and in vitro modeling of physiological systems. Other non-biological uses for this device include hydrology, mining, materials characterization, and applications that require local sampling/probing from an enclosed chamber.

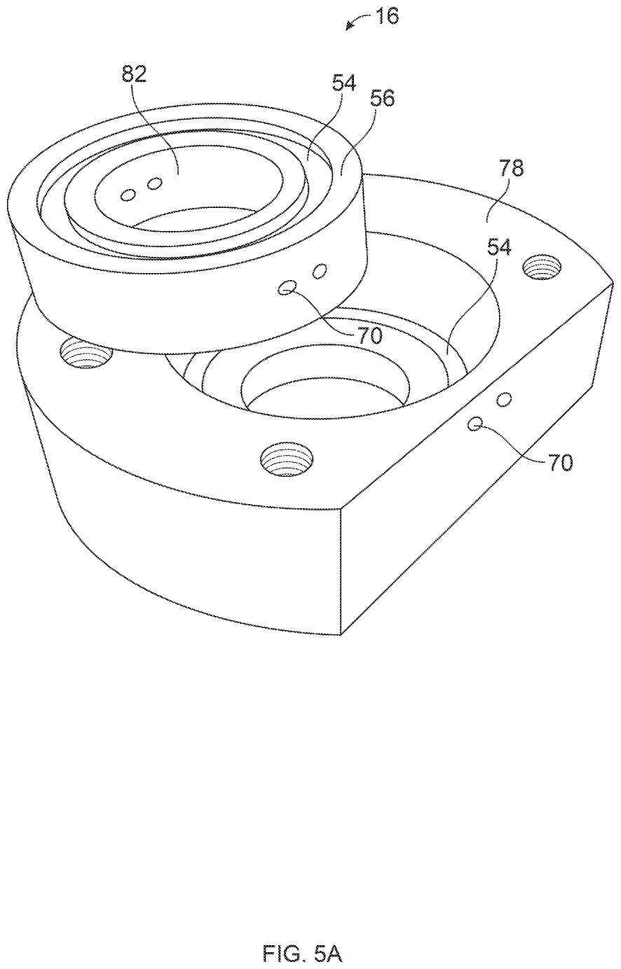

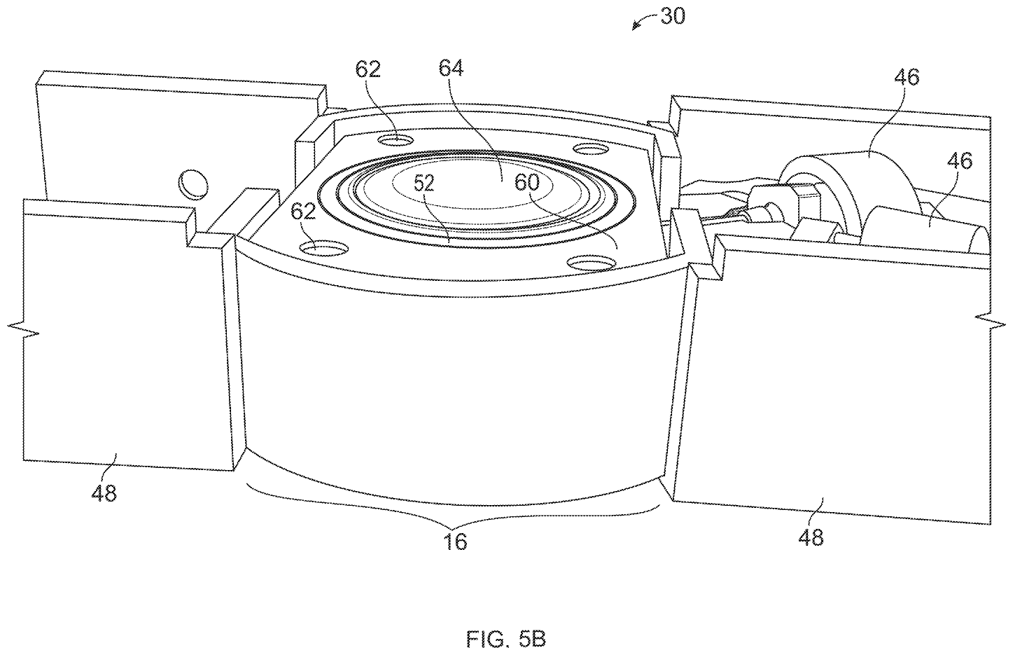

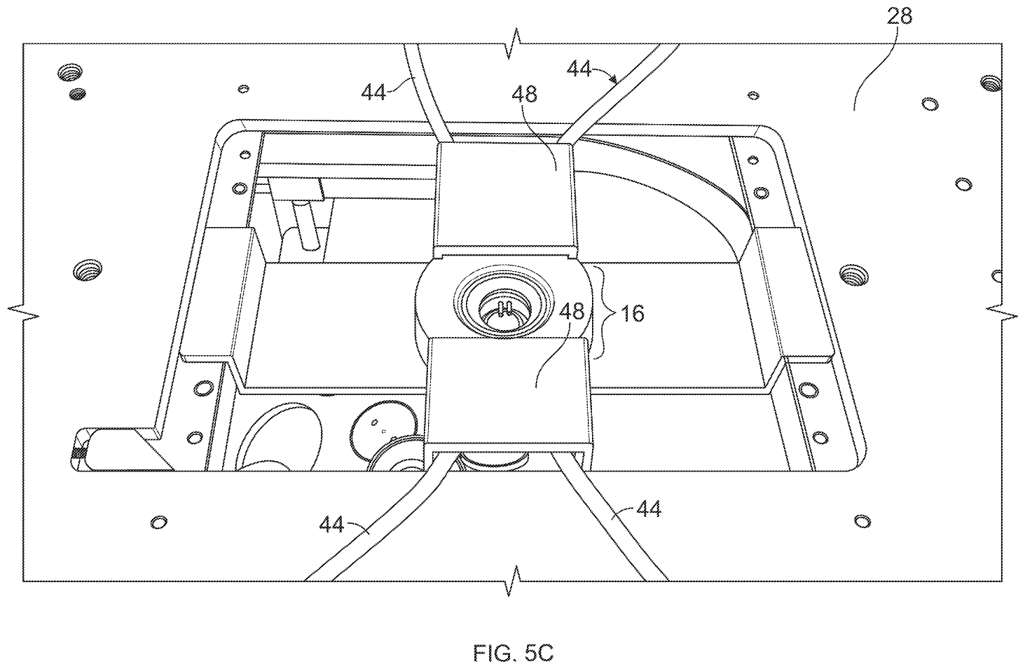

In one example, a custom designed Luer lock syringe track 44 and Luer lock holder 46 or syringe holders 80, in a track system 48 or an assembled platform 48, allows for controlled retraction of syringes 40 from the hydrogel 24. When the holders 80 are pushed toward the cell chamber 16 through the top portion 34, the bottom portion 36, an inner cylinder 56, or an outer cylinder 78, or multiple portions, the syringes 40 slide into the hydrogel 24 in the cell chamber, at least in accordance with one design (FIGS. 5A-5C). The top portion 34 may also be the inner cylinder 78, and the bottom portion 36 may be the outer cylinder of the cell chamber 16.

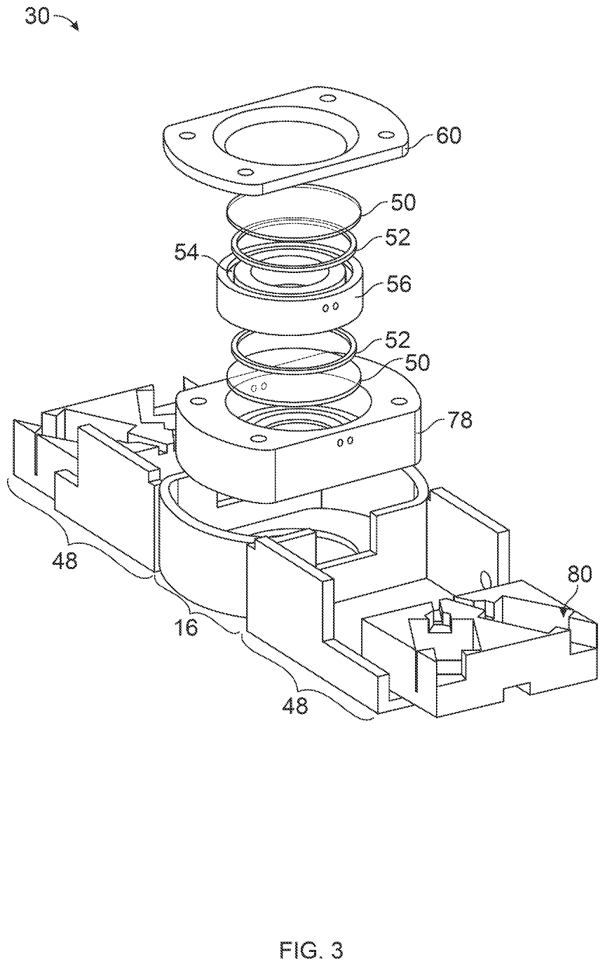

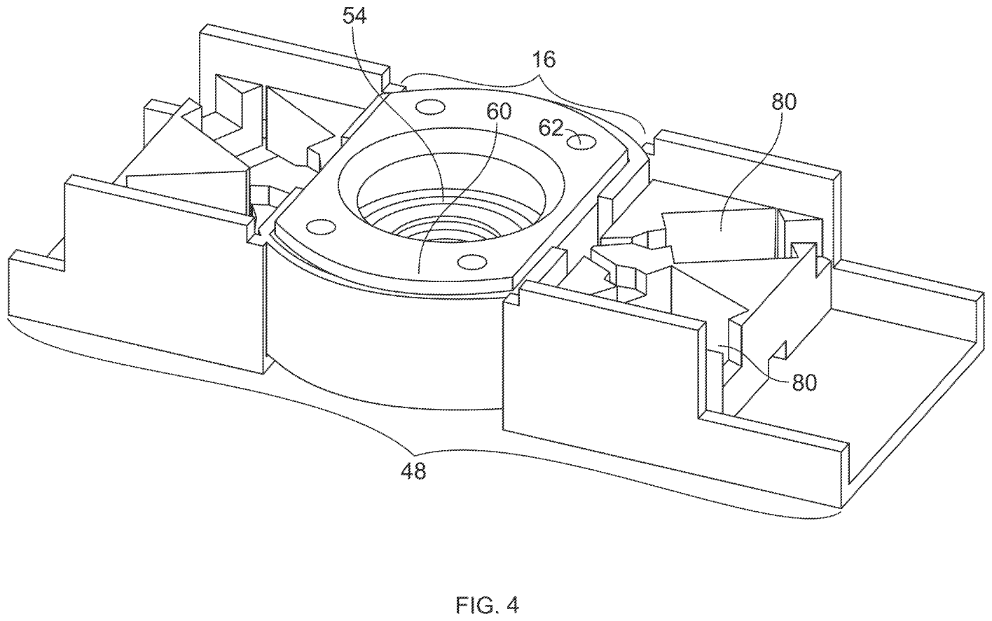

In one aspect, the cell chamber 16 that fits into the track system 48 is composed of multiple components (FIGS. 3-4) as follows: a coverslip 50 and sealing O-ring 52 fit into an inner slip 54, such as an aluminum slip. The inner cylinder 56 of the slip houses the desired hydrogel 24 also referred as the cell chamber in some aspects. The top of the aluminum slip 54 may be sealed with another O-ring 52 and coverslip 50. A top lid 60 may be bolted 62 at the four corners of the cell chamber 16 to compress the O-rings 52 for sufficient sealing. The cover slip may be made of translucent material. The platform 48 may comprise syringe holders 80 for guiding the syringe 40 into place.

One example of the disclosure is a fully assembled angiogenesis platform 48 with syringe holders 80 in position to penetrate the hydrogel 24. A hydrogel housing 64 is optimally disposed within the center cylindrical cavity 82 of the cell chamber 16. The assembled and inserted syringe configuration is consistent with appropriate position for hydrogel incubation period after injection into the cylindrical cavity (FIGS. 5A-5C).

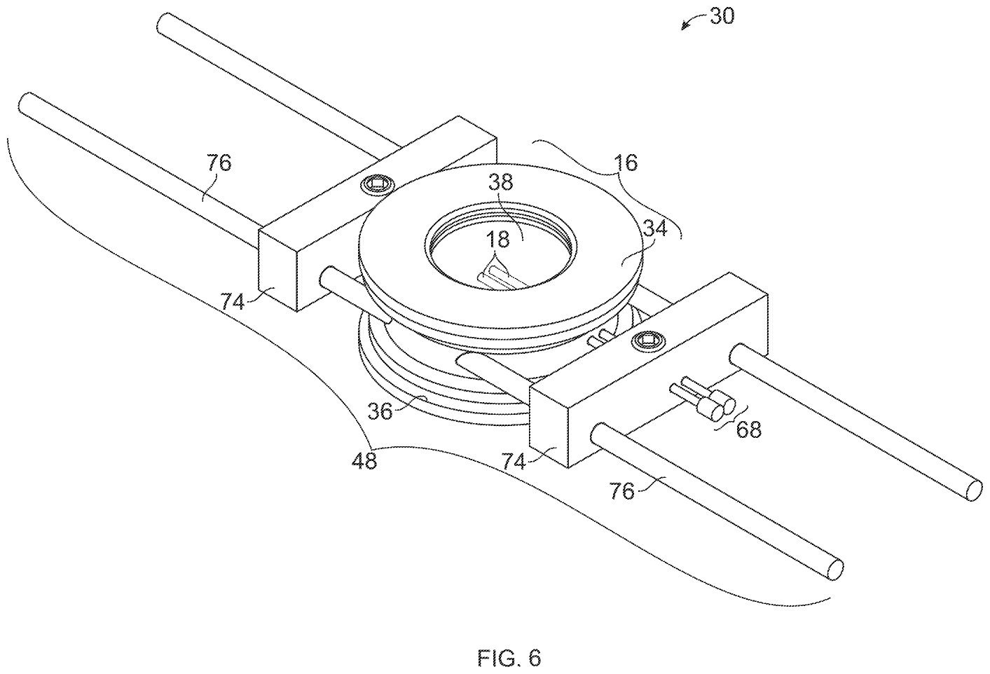

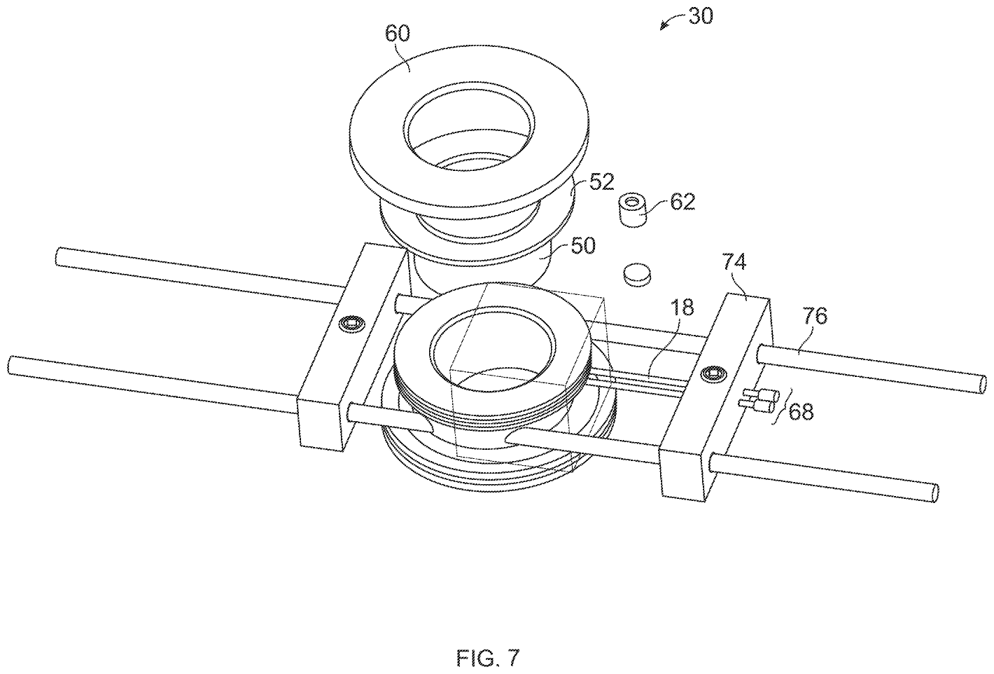

In vitro models (FIGS. 6 and 7) are valuable across a wide spectrum of biomedical research applications. In one aspect, an in vitro vascularized tissue model 30 with sustained physiological flow and a platform capable of accommodating various biomedical problems in nanoparticle exposure, drug delivery and tissue engineering are provided. Another aspect of the disclosure includes the potential inflammatory response endothelial cells exhibit when exposed to various concentrations of nanoparticles. Data revealed from these experiments may help to better understand the impacts on health from the inhalation of nanoparticles in ambient air.

According to at least one example, a device 30, system 10, and method of the present disclosure provides a platform 48 for setting a material into the cylindrical compartment 16 or cell chamber 16, having one or more perfusable channels 18, installing into a flow loop if necessary, sealing the compartment housing the material, and allowing for microscopic imaging. In at least one example, the device is machined out of aluminum or other metals. The device can include a low durometer material 52 (e.g., O-rings or silicone) to seal the inner housing compartment 56 and either bolts to apply compression or having the cylindrical compartments 56 and 78 come together around the soft durometer material 52 or O-rings 52 and fastened by threading 62. Another aspect includes, for example, one or more tracks 44 disposed to guide syringes 40 into and out of the cell chamber 16 that both set channels 18 in the material and act as part of the flow loop to pass fluid or other materials into and out of the cell chamber 16. These tracks 44 may be locked into place at a desired position to inhibit them from moving into or out of the cell chamber 16 when it is not intended. Additionally, glass coverslips 50 may be used on both the top and the bottom of the device to allow light to pass through when on a microscope 28 making the sealed device 30 microscope compatible while maintaining flow conditions. The distance between the syringe tracks 44 that pass through the device are at strictly 1 mm apart. In another aspect, the syringe tracks 44 are spaced apart to suit a specific in vitro vascularized tissue model. Depending on the desired application, one channel 18 can be removed by removing two of the syringes 44 resulting in one channel 18 that passes through the material as opposed to two parallel channels 18. Material compatible with device is only limited to material that can be cast into a cylindrical channel. In at least one example, a custom designed stage was 3D printed to secure the device 30 on the microscope stage and optimally configured for not interrupting flow in the device. Intended use of the device was modeled around biological systems and modeling in vivo systems more accurately in vitro. However, use of the device and methods of the present disclosure can be expanded to any system where flow occurs through some type of medium. Current and prospective use of this device and methods of the present disclosure may include, for example, setting biomimetic hydrogels 24 such as collagen and fibrin, seeding cells into the channel 18, and allowing them to attach and proliferate. It also includes introducing other materials like nanoparticles or drugs into the system and investigating the biological effects and delivery mechanisms. By removing one channel, aspects of the present disclosure can include, for example, investigating the embedding of organoids or spheroids into the hydrogel, effects of diffusion or interstitial flow of chemotactic agents through the hydrogel and effects of these agents on cells seeded in the channel perfusing the hydrogel.

FIG. 7 shows a partial deconstruction of an exemplary device 30 of the present disclosure, the top lid 60, the coverslip 50, the silicone seal 52, as well as the set screw 62 that is in place to lock the syringes in place on the tracks.

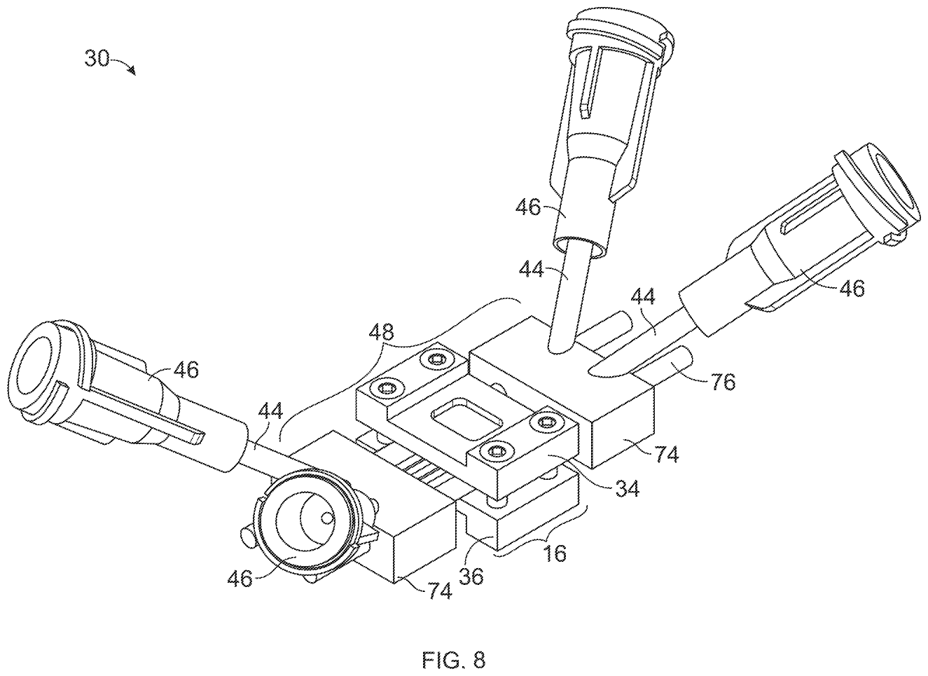

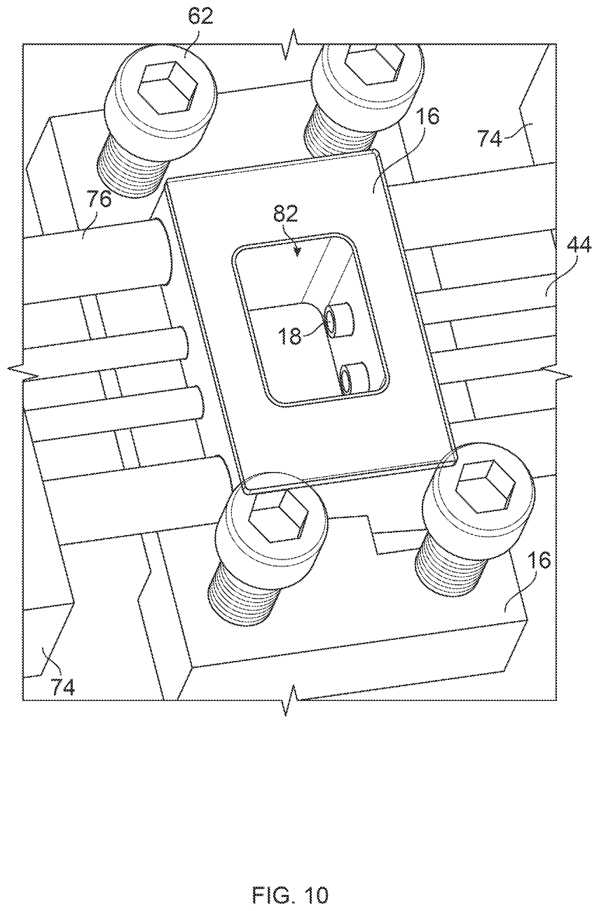

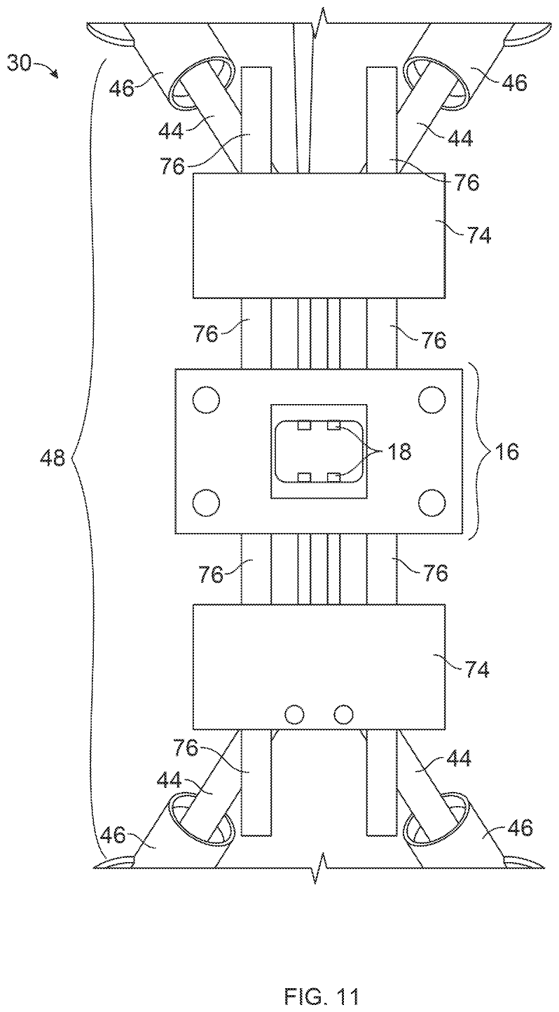

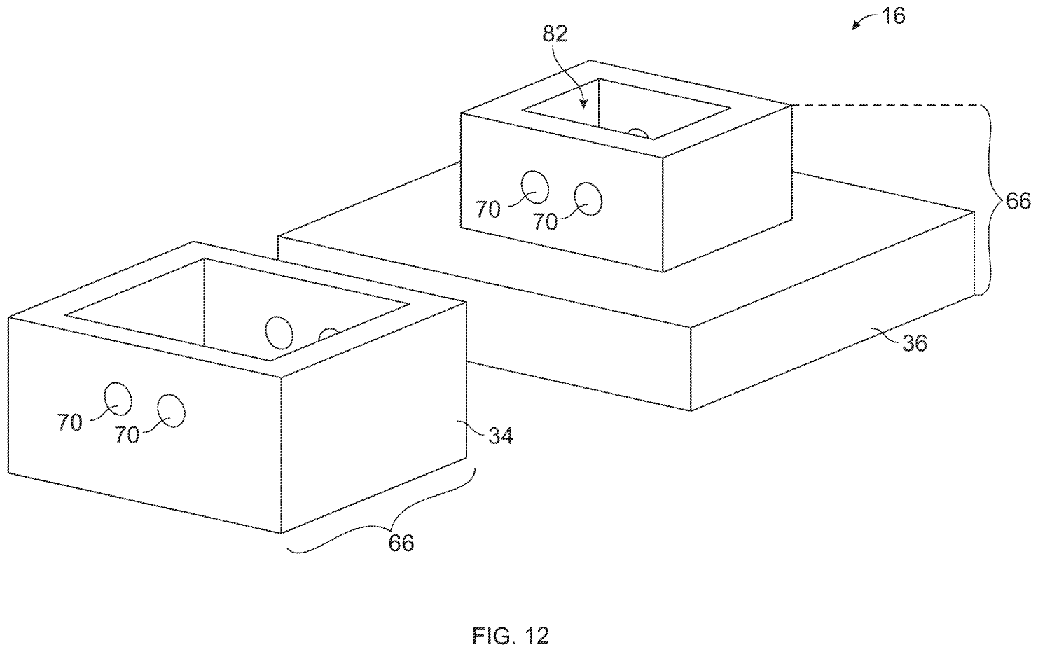

FIGS. 8-13 illustrate another aspect of the present disclosure. The chamber 16 may be constructed from a variety of engineering materials, such as aluminum, plexiglass, or thermoplastics that are used in 3D printing or injection molding. The shape of the chamber 16 may be cylindrical, square or rectangular. Outer dimensions of the body of the chamber 16 can range from smaller than 5 mm by 5 mm, depending on the limitations of the manufacturing process, to larger than 25 mm by 25 mm, depending on the spatial constraints of the imaging setup. The top 34 and bottom 36 are sealed with a transparent material 38, like glass, and they may be attached by press fitting, using screws to fasten down, or having a threaded lid.

The addition of the transparent material 38 through these orientations allows for imaging or microscopy for inspection of occurrences within the chamber 16. The chamber 16 has one or more holes 70 that pass through the body 66 from the side or top that allow tubing, syringes, or probes to pass into the chamber 16. The dimensions of the channels 18 can range from as small as 0.026 mm to as large as 2.1 mm in diameter, but these dimensions are simply limited by the outer geometry of the chamber 16. The channels 18 may run parallel or another orientation within as little as 0.5 mm apart, which is limited by the manufacturing process. The channels 18 can pass through the chamber 16 parallel to the base of the device, or they may have a slight angle.

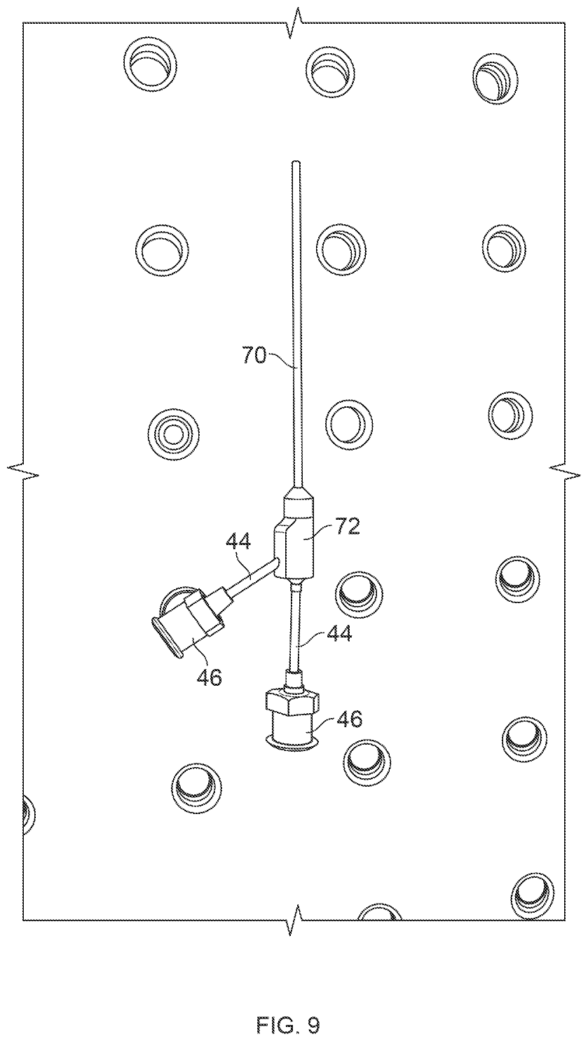

A significant and unique feature of this device 30 is that the channels 18 do not enter the chamber near a 90 degree angle like most other microfluidic models. Considering the angle of entry of the channels 18 through the body and chamber, a probe 40 of a smaller outer diameter can pass into the chamber to sample the local environment; to input an exogenous or additional material, chemical, or biological; or to provide some type of stimulation to the local environment within the channel 18. The tubing of the channel 18 is secured in place in the wall or cavity of the chamber that extends beyond the chamber 16, which allows attachment to a pump, access to a reservoir of fluid, and flow through the device. This specific design also provides access to gravity or pump driven flow into the chamber. Extensions 70 or connectors, as shown in FIG. 9, from this flow tubing in a Y, T junction 72, or other similar shape provide access to the flow stream and channel without disrupting the castable material or local environment that was developed within the chamber 16. The extension 70 or connector may be disposed in the track, slip, or platform for introducing one or more concentrations of nanoparticles into the cell chamber This geometrical junction point 72 is constructed through joining metal or polymeric tubing at the junction point, and we have also developed a rendition where the junction 72 occurs within a sliding block 74 that is external to the chamber 16 that slides along posts 76 that are attached to the chamber. The sliding posts 76 are oriented in a manner to allow attachment to the block 74 between them, where the tubing 70 may pass through the block 74 and allow for precise stabilization when moving the tubing 70 into or out of the center chamber 16, as well as locking the tubing 70 in place to avoid disruption from outward or inward movement within the chamber.

The chamber 16 may be attached to a stage on a microscope 28 for transient imaging under dynamic, static, or controlled flow conditions. The chamber 16 may be removed from the stage and incubated in another environment. Using manufacturing methods such as lithography, 3D printing, injection molding, or machining, the device can be replicated numerous times on a larger plate making numerous chambers in line with the other for high throughput applications.

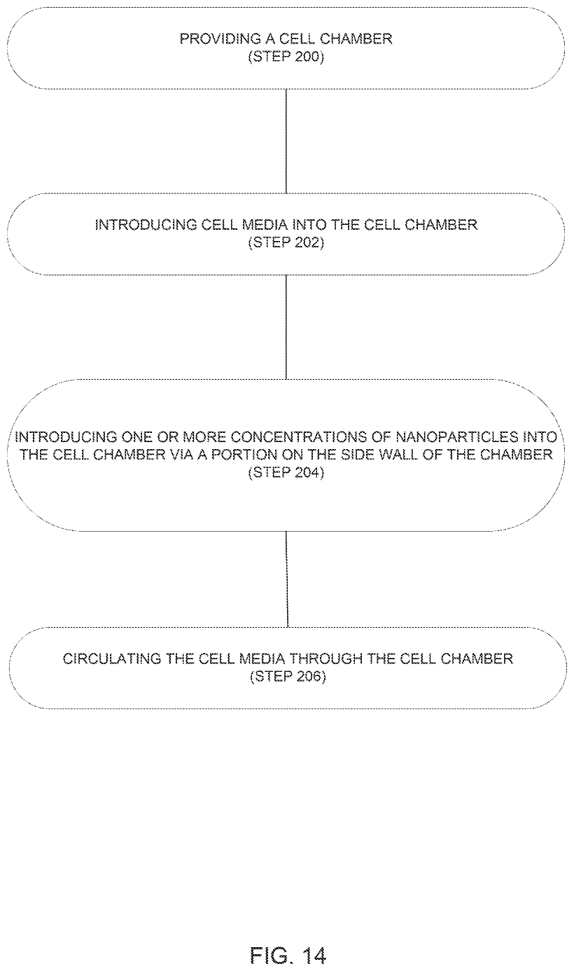

A method for physiologically-relevant, serum-free in vitro angiogenesis (FIG. 14) is disclosed. The method may include providing a cell chamber (Step 200). The cell chamber may have one or more perfusable channels, a viewing window for microscopy observation, and one or more ports in a sidewall of the cell chamber. The chamber may be on a track operably disposed on at least one side of the cell chamber or the chamber may be on a platform having a track operably disposed on at least one side of the cell chamber. Next, cell media may be introduced into the cell chamber (Step 202). The cell media may be introduced through channels. Next, one or more concentrations of nanoparticles may be introduced into the cell chamber (Step 204). The nanoparticles may be introduced via the one or more ports in the sidewall of the cell chamber. Syringes may be used to introduce the nanoparticles. The syringes may introduced the material into the hydrogel, housed in the cell chamber. The syringe may be moved by adjusting the position of the track. Lastly, the cell media is circulated through the cell chamber (Step 206). The syringe may be retracted from the hydrogel by adjusting the position on the track, before, after, or while the cell media is being circulated through the cell chamber. The peristaltic pump may be used to circulate the material through the cell chamber.

The invention is not to be limited to the particular embodiments described herein. In particular, the invention contemplates numerous variations in a physiologically-relevant, serum-free in vitro angiogenesis platform. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes or methods of the invention. It is understood that any other modifications, substitutions, and/or additions can be made, which are within the intended spirit and scope of the invention.

What is claimed is:

1. A physiologically-relevant, serum-free in vitro angiogenesis platform, comprising:
a cell chamber;
one or more perfusable channels disposed within the cell chamber;
one or more ports in a sidewall of the cell chamber;
a track operably disposed on at least one side of the cell chamber, and
at least one connector disposed in the track, the at least one connector configured for introducing one or more concentrations of nanoparticles into the cell chamber.

2. The platform of claim 1, further comprising:
a slip operably configured for housing the cell chamber.

3. The platform of claim 2, wherein the slip comprises the one or more ports.

4. The platform of claim 1, wherein the perfusable channels comprise hydrogel walls.

5. The platform of claim 1, further comprising:
one or more syringes for introducing the one or more concentrations of nanoparticles.

6. The platform of claim 5, wherein the syringe is operably locked by the at least one connector disposed on the track.

7. The platform of claim 6, wherein the syringe is inserted into a hydrogel in the cell chamber by adjusting the position of the track.

8. The platform of claim 5, wherein the syringe is retracted from a hydrogel in the cell chamber by adjusting the position of the track.

9. A system for a physiologically-relevant, serum-free in vitro angiogenesis platform, comprising:
an incubator operably configured for housing a cell media;
a peristaltic pump for moving the cell media;
a microscope for conducting microscopy of the cell media; and
a cell chamber having one or more perfusable channels, a viewing window for observation with the microscope, and one or more ports in a sidewall of the cell chamber for introducing one or more concentrations of nanoparticles;
wherein endothelial cells line the one or more perfusable channels.

10. The system of claim 9, further comprising a slip having one or more ports in operable communication with the one or more ports in the sidewall of the cell chamber.

11. The system of claim 10, further comprising:
one or more syringes for introducing the one or more concentrations of nanoparticles.

12. The system of claim 10, further comprising:
a track operably disposed on at least one side of the cell chamber.

13. The system of claim 12, further comprising:
at least one connector disposed in the track, the at least one connector configured for introducing one or more concentrations of nanoparticles into the cell chamber.

14. The system of claim 10, wherein the syringe is adjusted in position relative to the cell media in the cell chamber by adjusting the position of the track.

15. A method for physiologically-relevant, serum-free in vitro angiogenesis, comprising:
providing a cell chamber having one or more perfusable channels, a viewing window for microscopy observation, and one or more ports in a sidewall of the cell chamber;
introducing cell media into the cell chamber;
introducing one or more concentrations of nanoparticles into the cell chamber via the one or more ports in the sidewall of the cell chamber; and
circulating cell media through the cell chamber.

16. The method of claim 15, further comprising:
a track operably disposed on at least one side of the cell chamber.

17. The method of claim 15, further comprising:
introducing the one or more concentrations of nanoparticles with one or more syringes.

18. The method of claim 15, further comprising:
inserting a syringe into a hydrogel in the cell chamber by adjusting the position of a track.

19. The method of claim 15, further comprising:
retracting the syringe from a hydrogel in the cell chamber by adjusting the position of the track.

20. The method of claim 16, wherein the track comprises at least one connector configured for introducing one or more concentrations of nanoparticles into the cell chamber.
